(19) United States
(12) Patent Application Publication
(10) Pub. No.: US 2008/0286390 A1
(43) Pub. Date: Nov. 20, 2008

(54) Skin care composition

(76) Inventor: Judy Tanyi, Smyrna, GA (US)

Correspondence Address: LITMAN LAW OFFICES, LTD., P.O. BOX 15035, CRYSTAL CITY STATION, ARLINGTON, VA, 22215-0035, US

(21) Appl. No.: 12/149,153

(22) Filed: Apr. 28, 2008

Related U.S. Application Data
(60) Provisional application No. 60/924,480, filed on May 16, 2007.

(51) Int. Cl. A61K 36/886; A61K 31/045; A61K 36/63; A61Q 19/00

(52) U.S. Cl. 424/744; 514/729; 424/725

(57) ABSTRACT

The skin care composition is a cream or lotion that moisturizes the skin, relieving dryness, itchiness, and eczema. The skin care composition contains moisturizers and a plurality of natural oils containing essential fatty acids, the natural oils being selected from the group consisting of borage oil, omega-3 oil, emu oil, evening primrose oil, safflower oil, hemp seed oil, flaxseed oil, and rosehip oil. The moisturizers include olive oil and glycerin. In addition, the skin care composition contains an anti-inflammatory agent, preferably aloe vera juice and aloe extract, and emulsifiers, stabilizers, emollients for softening the skin, and water. The skin care composition may also contain a fragrance and a preservative. Optionally, the skin care composition may contain menthol crystal.

### CROSS-REFERENCE TO RELATED APPLICATION

This application claims the benefit of U.S. Provisional Patent Application Ser. No. 60/924,480, filed May 16, 2007.

### BACKGROUND OF THE INVENTION

1. Field of the Invention

The present invention relates to skin care products, and particularly to a skin care composition that moisturizes the skin, relieving dryness, itchiness, and eczema.

2. Description of the Related Art

Proper care of the skin is an important component of good hygiene. While bathing and washing one's face and hands are essential to proper skin care, quite often soap is not enough. Commercial soaps are particularly formulated to remove dirt and oils from the body, and they are usually quite effective in doing so, but they are often harsh, leaving the skin dry. Often bar soaps will have certain additives to enhance advertising to attract the consumer, such as a fragrance, a deodorant, etc. A few claim to have moisturizers, but many people fail to see any significant improvement after using such bar soaps.

Dry skin has many causes, from exposure to sun and wind to diet to exposure to bacteria, fungus, too much washing with soap, and the like. Dry skin is often rough to the touch, and may cause itching or irritation. In some cases, the irritation may lead to excessive scratching, redness, tiny papules and vesicles, scaling, and sometimes changes in pigmentation, all telltale signs of eczema. Consequently, many people turn to various skin lotions for the relief of skin irritation, to improve the appearance of their skin, and particularly to moisturize dry skin.

Although organic chemists have been successful in isolating various compounds and active ingredients beneficial to the skin, and subsequently synthesizing such compounds from simpler compounds in the laboratory, many people feel a higher level of comfort using skin care products that are formulated primarily from substances that have been formed or extracted from natural botanical or animal products. Such formulations are often felt to have fewer adverse skin reactions, to have enhanced effectiveness through synergy between the “active ingredient” and other related compounds native to the natural product, and to contain ingredients that have acquired a reputation for effectiveness through custom, folklore, anecdotal evidence, and through a few scientific studies by doctors of natural medicine and related health care practitioners.

Nevertheless, it is generally acknowledged that there is no one single effective substance or remedy for dry skin, so that every formulation depends upon combinations of different natural products. Further, due to the different causes for dry skin and the differences in each person's genetic makeup, it may be that different combinations of natural ingredients are beneficial in moisturizing dry skin for different groups of people. Thus, a skin care composition solving the aforementioned problems is desired.

### SUMMARY OF THE INVENTION

The skin care composition is a cream or lotion that moisturizes the skin, relieving dryness, itchiness, and eczema. The skin care composition contains moisturizers and a plurality of natural oils containing essential fatty acids, the natural oils being selected from the group consisting of borage oil, omega-3 oil, emu oil, evening primrose oil, safflower oil, hemp seed oil, flaxseed oil, and rosehip oil. The moisturizers include olive oil and glycerin. In addition, the skin care composition contains an anti-inflammatory agent, preferably aloe vera juice and aloe extract, and emulsifiers, stabilizers, emollients for softening the skin, and water. The skin care composition may also contain a fragrance and a preservative. Optionally, the skin care composition may contain menthol crystal.

The skin care composition is formulated as a cream or lotion having a butter-like consistency. When applied topically, the composition moisturizes the skin, relieves itching and inflammation, and may aid in reducing lesions associated with skin ailments, such as eczema.

These and other features of the present invention will become readily apparent upon further review of the following specification.

### DETAILED DESCRIPTION OF THE PREFERRED EMBODIMENTS

The present invention is a skin care composition formulated as a cream or lotion that moisturizes the skin, relieves itching and inflammation, and may aide in reducing lesions associated with skin ailments, such as eczema.

It has been noted that eczema is associated with a deficiency in essential fatty acids, i.e., fatty acids necessary for the maintenance of good health, but which the body is unable to produce naturally. Therefore, essential fatty acids must be provided in the diet. Essential fatty acids include linoleic acid (an omega-6 fatty acid) and alpha-linolenic acid (an omega-3 fatty acid). The body cannot produce omega-6 or omega-3 fatty acids, since it is unable to produce a carbon-carbon double bond more than nine carbon atoms from the carboxylic acid end of the molecule. Omega-6 and omega-3 fatty acids are precursors to the production of prostaglandins, which help to regulate the body's inflammatory response via the formation of arachidonic acid and eicosapentaenoic acid.

Whether the deficiency of essential fatty acids is due to dietary insufficiency or due to a deficiency in the enzymes necessary to convert essential fatty acids to prostaglandins resulting from stress, bacterial infection, or other causes is an open question. While some skin care formulations include one or another of the essential fatty acids, it has been found that a skin care formulation that is effective for one person may not be effective for another person. Without wishing to be bound by theory, the skin care composition of the present invention may derive its effectiveness from providing a plurality of essential fatty acids of different types derived from a plurality of natural sources, coupled with moisturizers, emollients for softening the skin, and an anti-inflammatory agent. The essential fatty acids are readily transported through the skin cells to remedy the fatty acid deficiency without blocking the skin pores, while the oils and emollients provide humectants to attract moisture to the skin and an occlusive effect to prevent moisture from escaping the skin.

The skin care composition contains moisturizers, including olive oil and glycerin, which act as humectants to increase the capacity of the stratum corneum, or outer layer of the skin, to retain moisture. Moisturizers help to relieve dryness of the skin, which can lead to itching.

The skin care composition contains a plurality of essential fatty acids derived from natural oils, which include both botanicals and oils derived from animal sources. The natural oils are selected from the following: borage oil, which contains about 60% omega-6 fatty acids, including about 24% gamma-linolenic acid, an intermediate produced in the metabolism of linoleic acid to arachidonic acid; omega-3 oil, which may be derived from a variety of sources, most commonly from fish oil; emu oil, derived from the emu bird and which contains 70% unsaturated fatty acids, the majority of which is oleic acid (an omega-9 fatty acid), but also includes 20% linoleic acid and ½% omega-3 fatty acids; evening primrose oil, which contains 70% omega-6 fatty acids, including 10% gamma-linolenic acid; safflower oil, which contains 70% linoleic acid; hemp seed oil, which contains 60% omega-6 and 20% omega-3 fatty acids; flaxseed oil, which contains 40-60% alpha-linolenic acid and about 15% linoleic acid; and rosehip oil, which contains about 45% linoleic acid and 32% linolenic acid.

The skin care composition contains at least one stabilizer to prevent decomposition or degradation of the essential fatty acids. Preferably, the stabilizer is vitamin E, a powerful antioxidant that prevents the oxidation of the fatty acids, which might result in the formation of lipid peroxides.

The skin care composition contains an anti-inflammatory agent, preferably aloe vera juice and aloe extract. Aloe vera is well known for its anti-inflammatory activity, which reduces itching and redness.

The skin care composition includes emollients for softening the skin. The emollients may include jojoba oil, shea butter, and cocoa butter (cocoa butter is optional, and may be substituted for by additional olive oil).

The skin care composition contains emulsifiers, such as emulsifying wax (vegetable-based emulsifying waxes typically include at least cetearyl alcohol and polysorbate 60) and stearic acid. The skin care composition also includes water, which is preferably distilled, de-ionized, or otherwise purified water.

The skin care composition also may optionally include menthol crystal, which is a compound derived from peppermint oil or other mint oils. The menthol provides a cooling sensation to the skin, serving as a counterirritant to reduce itching. Also, a preservative, such as Germaben® II-E (Germaben is a registered trademark of Sutton Laboratories, Inc. of Chatham, N.J.), which contains a soluble mixture of methylparaben, propylparaben, and other parabens in propylene glycol, and fragrances may be added, as desired.

The skin care composition preferably contains at least one natural oil having a high omega-6 fatty acid content, i.e., a concentration of at least 10% omega-6 fatty acid, and at least one natural oil having a high omega-3 content, i.e., a concentration of at least 10% omega-3 fatty acid. The natural oils may be derived by extraction, by distillation, by pressing, by maceration, or by other processes known for obtaining natural vegetable, herbal, animal-based, or essential oils.

An example of a skin care composition according to the present invention may be made from the ingredients listed in Table I below.

TABLE I
SAMPLE SKIN CARE COMPOSITION

| Component | Quantity |
|---|---|
| Distilled water | 4 oz.-118.28 ml |
| Aloe vera juice | 28 oz.-827.97 ml |
| Emulsifying wax | 1.5 oz.-43.53 g |
| Stearic acid | 3.5 oz.-42.53 g |
| Shea butter | 3.5 oz.-99.23 g |
| Cocoa butter | 1 oz.-28.35 g |
| Evening primrose oil | 2.5 oz.-73.93 ml |
| Emu oil | 4 oz.-118.28 ml |
| Rosehip oil | 2 tbs-29.57 ml |
| Vitamin E | 2 tbs-29.57 ml |
| Jojoba oil | 3 tbs-44.53 ml |
| Borage oil | 3 tbs-44.53 ml |
| Hemp oil | 1 tsp-4.93 ml |
| Olive oil | 2 ox-59.14 ml |
| Safflower oil | 1 tsp-4.93 ml |
| Aloe extract | ½ tsp-2.46 ml |
| Flaxseed oil | 1 tsp-4.93 ml |
| Omega-3 extract | ½ tsp-2.46 ml |
| Glycerin | ½ tsp-2.46 ml |

The ingredients in Table I are all measured. The emulsifying wax, stearic acid, and olive oil are combined in a saucepan over a double boiler on a low heat and stirred as the wax melts at between 110-120° F., taking care that the temperature does not exceed 120° F. to avoid thermal degradation of the oil. Shea butter and, optionally, cocoa butter are added to the mixture, and heating is continued until melting is complete, and then the remaining oils (emu, jojoba, borage, rosehip, hemp [optional], evening primrose, safflower [optional], and flaxseed [optional]) are added, and the mixture is removed from the heat. Menthol crystals may optionally be added with the remaining oils, if desired, and allowed to dissolve in the mixture of oils before removing the oils from the heat. The distilled water, aloe vera juice, and aloe extract are brought to a boil in a separate pot, removed from the heat, and cooled for a few minutes. The water and aloe components are slowly poured into the melted oils and mixed with a blender. As the mixture cools, it thickens, and the color changes to white. Vitamin E and glycerin are added while continuing mixing, and a fragrance (orange oil, lemon oil, etc.) and a preservative, such as Germaben II-E, are added. The mixture is poured into clean containers for storage and use.

The following examples illustrate use of the composition.

### Example 1

The composition was used to treat eczema on a four-year old female. The eczema was on the neck, legs, shoulders, and a large portion of the stomach. The eczema had been recurring for a period of two years and caused itching, especially at night. Upon topical application of the cream to the affected area two to three times per day, the itching stopped within the first two days. Within one week, the patches on the skin started to smooth out. Within three weeks, the appearance of the skin had improved to the point that the subject was able to wear clothes that left the affected areas exposed without embarrassment.

### Example 2

The composition was used to treat eczema in a six-year old female with a severe case of long standing. The subject's arms were particularly affected, requiring the subject to wear long sleeves, and sleeping was adversely affected by nocturnal itching. The skin care composition was topically applied 3-4 times daily. Within a few days, the subject was able to sleep through the night without discomfort. After use for one month, the scaliness and patches were gone from the arms, and the subject was able to wear short sleeves without embarrassment.

### Example 3

An adult female, employed as a nurse, had very dry skin that showed some cracks in the skin. The subject had tried numerous creams and lotions without success. The skin care composition was applied 2-3 times per day. Within one week, the skin was noticeably softer, rejuvenated, hydrated, and the dry, crackly skin had smoothed out and acquired the tone of new skin.

It is to be understood that the present invention is not limited to the embodiments described above, but encompasses any and all embodiments within the scope of the following claims.

I claim:

1. A skin care composition, comprising:
a plurality of natural oils containing essential fatty acids, the oils being selected from the group consisting of borage oil, omega-3 oil, emu oil, evening primrose oil, safflower oil, hemp seed oil, flaxseed oil, and rosehip oil;
a moisturizer;
a skin-softening emollient;
an anti-inflammatory agent;
a stabilizer;
an emulsifier; and
purified water.

2. The skin care composition according to claim 1, wherein the formulation is formed as a cream.

3. The skin care composition according to claim 1, wherein the formulation is formed as a lotion.

4. The skin care composition according to claim 1, further comprising menthol crystals.

5. The skin care composition according to claim 1, wherein the skin softening emollient is selected from the group consisting of jojoba oil, shea butter, and cocoa butter.

6. The skin care composition according to claim 1, wherein the anti inflammatory agent comprises aloe Vera juice and aloe extract.

7. The skin care composition according to claim 1, wherein the stabilizer comprises vitamin E.

8. The skin care composition according to claim 1, wherein the emulsifier comprises vegetable-based emulsifying waxes, including at least cetearyl alcohol, polysorbate 60, and stearic acid.

9. The skin care composition according to claim 1, wherein the purified water comprises distilled water.

10. The skin care composition according to claim 1, wherein the plurality of natural oils contain at least one natural oil having a concentration of at least 10% omega-6 fatty acid, and at least one natural oil having a concentration of at least 10% omega-3 fatty acid.

11. A skin care composition, comprising:
a plurality of natural oils containing essential fatty acids, the plurality of oils including at least one natural oil having a high omega-6 fatty acid content and at least one natural oil having a high omega-3 fatty acid content;
a moisturizer;
a skin-softening emollient;
an anti-inflammatory agent;
a stabilizer;
an emulsifier; and
purified water.

12. The skin care composition of claim 11, wherein the formulation is formed as a cream.

13. The skin care composition of claim 11, wherein the formulation is formed as a lotion.

14. The skin care composition of claim 11, further comprising menthol crystal.

15. The skin care composition of claim 11, wherein the moisturizer comprises olive oil and glycerin.

16. The skin care composition of claim 11, wherein the skin-softening emollient is selected from the group consisting of jojoba oil, shea butter, and cocoa butter.

17. The skin care composition of claim 11, wherein the anti-inflammatory agent comprises aloe Vera juice and aloe extract.

18. The skin care composition of claim 11, wherein the emulsifiers comprise emulsifying wax and stearic acid.

19. A skin care composition, comprising:
a plurality of natural oils containing essential fatty acids, the oils including borage oil, omega-3 oil, emu oil, evening primrose oil, safflower oil, hemp seed oil, flaxseed oil, and rosehip oil;
moisturizers, including olive oil and glycerin;
skin-softening emollients, including jojoba oil, shea butter, and cocoa butter;
aloe vera juice and aloe extract for anti-inflammatory effect;
vitamin E for stabilizing the essential fatty acids;
emulsifiers, including emulsifying wax and stearic acid; and
distilled water.
